(19) United States
(12) Patent Application Publication
(10) Pub. No.: US 2001/0055540 A1
(43) Pub. Date: Dec. 27, 2001

(54) METHODS OF USING AN AIR ENHANCING DISC AND A HAIR DRYER

(76) Inventor: EMMETT J. BONAMARTE, HIGHLAND PARK, IL (US)

Correspondence Address: STEPHEN G RUDISILL, JENKENS & GILCHRIST, 1445 ROSS AVENUE, SUITE 3200, DALLAS, TX, 75202-2799, US

(21) Appl. No.: 09/240,380

(22) Filed: Jan. 29, 1999

(51) Int. Cl. A61L 9/00

(52) U.S. Cl. 422/4; 422/120

(57) ABSTRACT

A method of using an air enhancing disc and a hair dryer. The hair dryer has a cold air inlet and an air outlet. In one embodiment, the air enhancing disc has a plurality of holes for allowing passage of air therethrough. The air enhancing disc has a first surface and a second surface. The air enhancing disc is releasably engaged to an outer surface of the cold air inlet. Air is allowed through the plurality of holes and through the cold air inlet. A stream of enhanced air is released from the air outlet of the hair dryer. The air enhancing disc may include perfume, cologne, scents, deodorant, or combinations thereof. Alternatively, the air enhancing disc may include vitamins or materials adapted to assist in stimulating growth or regeneration of hair.

### FIELD OF THE INVENTION

The present invention is directed to methods of using an air enhancing disc and a hair dryer. In particular, the present invention is directed to an air enhancing disc that is scented and/or supplemented with vitamins.

### BACKGROUND OF THE INVENTION

The use of scented or fragranced materials for hair has been popular and desirable for some time. These scented or fragranced materials may come from a variety of sources. Scented or fragranced materials may be in the form of sprays that are applied by, for example, plastic containers with small pumps or aerosol containers. The use of hair dryers for drying hair has also been popular for many years, with hair dryers being used in various locations, from hair salons and health clubs to individual dwellings.

In the past, drying and scenting the hair have typically been performed in separate and distinct steps. There have been processes, however, which combined drying and scenting the hair. These existing processes have disadvantages, such as requiring purchasing of additional special hair drying attachments and/or the need to replace existing components of the hair dryer with special assemblies. These disadvantages may result in limiting the existing processes to certain hair dryer models and/or adding complicated steps for a user in at least the initial setup. At the very least, the purchase of additional attachments and/or replacement of existing components results in additional costs being incurred by the user. Another drawback of existing processes may include reduction of air flow from the outlet of the hair dryer. This may result in other problems, including damage or failure of the fan motor.

Accordingly, a need exists for methods of drying and enhancing the hair at the same time that overcomes the above-noted shortcomings associated with existing processes.

### SUMMARY OF THE INVENTION

One method of the present invention includes the use of an air enhancing disc and a hair dryer. The hair dryer has a cold air inlet and an air outlet. The air enhancing disc is provided with means for allowing passage of air therethrough. The air enhancing disc has a first surface and a second surface, and is releasably engaged to an outer surface of the cold air inlet. Air is allowed through the means for allowing passage of air in the disc and through the cold air inlet. A stream of enhanced air is released from the air outlet of the hair dryer. The air enhancing disc may include perfume, cologne, scents, deodorant, or combinations thereof. Alternatively, the air enhancing disc may include vitamins or materials adapted to assist in stimulating growth or regeneration of hair.

### BRIEF DESCRIPTION OF THE DRAWINGS

Other objects and advantages of the invention will become apparent upon reading the following detailed description and upon reference to the drawings.

FIG. 1 is a front view of an air enhancing disc according to one embodiment that is to be used in a method of the present invention.

FIG. 2 is a front view of an air enhancing disc according to another embodiment that is to be used in a method of the present invention.

FIG. 3 is a cross-sectional view of the air enhancing disc of FIG. 1 taken generally across line 3-3 of FIG. 1.

FIG. 4 is a cross-sectional view of the air enhancing disc of FIG. 2 taken generally across line 4-4 of FIG. 2.

FIG. 5 is a front view of an air enhancing disc according to another embodiment that is to be used in a method of the present invention.

FIG. 6 is an exploded view of a conventional hand-held hair dryer and an air enhancing disc similar to FIG. 1 to be used according to one method of the present invention.

While the invention is susceptible to various modifications and alternative forms, a specific embodiment thereof has been shown by way of example in the drawings and will herein be described in detail. It should be understood, however, that it is not intended to limit the invention to the particular forms disclosed but, on the contrary, the intention is to cover all modifications, equivalents, and alternatives falling within the spirit and scope of the invention as defined by the appended claims.

### DESCRIPTION OF ILLUSTRATIVE EMBODIMENTS

Air Enhancing Discs

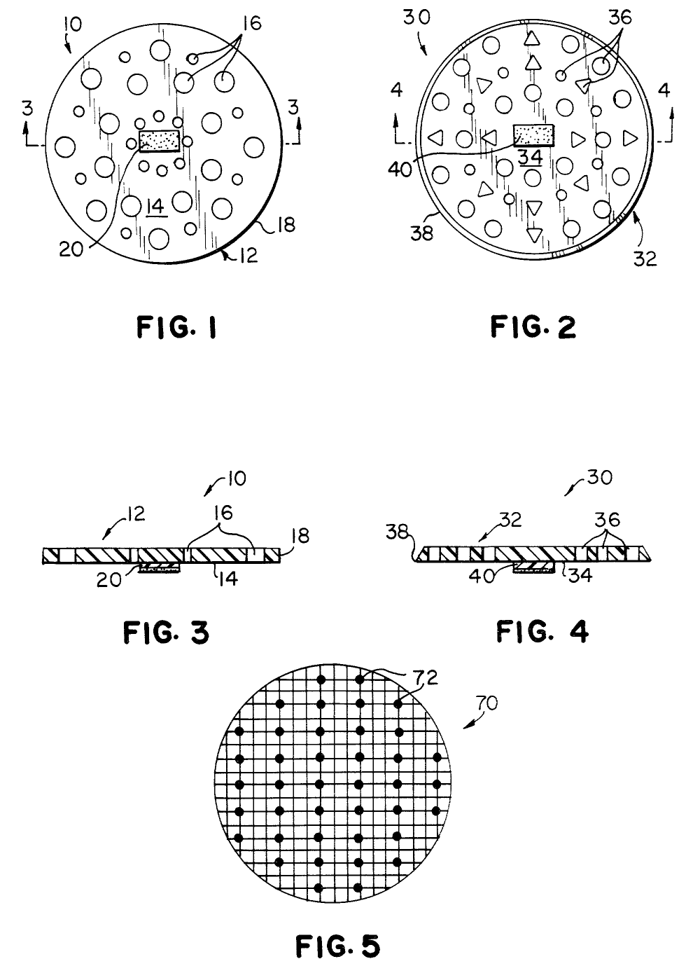

An air enhancing disc 10 according to one embodiment that is to be used in a method of the present invention is shown in FIG. 1. Other embodiments of an air enhancing disc 30 and 70 are shown in FIGS. 2 and 5, respectively. Referring back to FIG. 1, the air enhancing disc 10 includes a first surface 12 and a second surface 14. The air enhancing disc 10 is shown in FIG. 3 as having side walls 18 that extend generally perpendicular from the first surface 12 to the second surface 14. The air enhancing disc 30 as shown in FIG. 4, however, has side walls 38 that taper outwardly from a first surface 32 to a second surface 34.

The air enhancing disc 10 of FIG. 1 includes means for allowing an air stream to pass therethrough. According to one embodiment of the present invention, a plurality of holes 16 are formed in the air enhancing disc 10 of FIG. 1. The plurality of holes 16 of FIG. 1 are preferably sized and shaped so as to inhibit any potential damage to the hair dryer caused by a reduced flow of cold air through the cold air inlet or intake. A significant reduction of air through the cold air inlet may result in damage to or failure of the fan motor. According to another embodiment, a permeable disc may be used that would allow a cold air stream to pass therethrough.

It is also contemplated that a single large hole (not shown) may be formed in the air enhancing disc to allow a cold air stream to pass therethrough and also to permit hair enhancing material, such as scented or fragranced material and/or vitamins, to be released therefrom. The vitamins may include materials to improve the aesthetic appeal of hair, such as for shiny or bouncy hair, hair growth, or regeneration.

According to one embodiment, as shown in FIG. 1, the plurality of holes 16 are generally circular. The plurality of holes 16 may be of different geometric shapes, such as oval, triangular, square, rectangular, hexagonal, polygonal, and the like. For example, in FIG. 2, a plurality of holes 36 include generally circular-shaped holes and generally triangular-shaped holes. The plurality of holes may also be of different sizes that may vary between individual air enhancing discs and even within an individual air enhancing disc (see, e.g., FIG. 1).

The air enhancing discs are adapted to releasably engage a cold air inlet of a hair dryer. Specifically, for example, the second surface 14 of FIG. 1 is adapted to releasably engage the cold air inlet of a hair dryer. To assist in releasably engaging the cold air inlet, the air enhancing disc of FIGS. 1 and 2 include at least one doubled-sided tape 20 or 40, respectively, attached thereto. A first side of the double-sided tape may be attached to the air enhancing disc in a variety of locations, including being placed in the approximate center of the disc as shown in FIGS. 1 and 2. A second side of the double-sided tape is adapted to detachably engage to an outer surface of the cold air inlet. The second side of the double-sided tape may include a peel off strip or the like to inhibit portions of the tape from attaching to other portions of the tape. The air enhancing disc may include a single-sided tape (not shown) that is attached to the disc. The single-sided tape may be attached to the disc by an adhesive or the like. In another embodiment, the air enhancing disc may use an adhesive backing or the like to attach to an outer surface of the cold air inlet.

According to another embodiment, the air enhancing disc may be detachably engaged to an outer surface of the cold air inlet with a clip-on structure (not shown). The clip-on structure may be attached to the air enhancing disc and adapted to be placed into the cold air inlet. Alternatively, the clip-on structure may be located on the hair dryer itself.

According to yet another embodiment, the air enhancing disc may releasably engage the outer surface of the cold air inlet solely by the pressure being exerted on a surface. In FIG. 1, for example, the pressure would be exerted on the second surface 14. Of course, this pressure or suction would typically occur during operation of the hair dryer.

The air enhancing disc may include scented or fragrant material therein adapted to be released into the air when contacting passing air. The scented or fragrant material is intimately contacted with the air and additionally intensified by the heating element of a hair dryer. For example, the scented or fragrant material may include perfume, cologne, scents, deodorant, combinations thereof, and the like. The scented or fragrant material may, of course, be selected to appeal to certain individuals. According to another embodiment, the air enhancing disc may include materials, such as vitamins, to improve the aesthetic appeal of hair. The air enhancing disc may also include material adapted to assist in stimulating the growth or regeneration of hair. The hair enhancing materials, as well as the fragrant materials, are adapted to be released into the air when contacting a passing cold air stream, and may be combined with the above-described fragrant material.

The air enhancing material may be filtered, soaked, absorbed, or the like into, for example, the air enhancing disc 10. In one process, the air enhancing disc 10 may be dipped into a container of liquid material for a period sufficient for the material to absorb therein. The air enhancing material may be an oil-based liquid material or an alcohol-based liquid material. Oil-based liquid materials are generally preferred over alcohol-based liquid materials since oil-based materials are typically more resistant to evaporation and, thus, will last longer. Of course, the material cannot resist evaporation to an extent that the material will not be released into the cold air passing through the cold air inlet of a hair dryer. Other types of air enhancing material that generally resist evaporation are contemplated in the present invention.

The air enhancing disc used in the methods of the present invention may be made from a variety of materials, which are preferably flexible to adapt to varying styles, shapes and sizes of cold air inlets in hair dryers. In addition, the material is preferably durable so as to remain intact when subject to the stream of cold air passing therethrough. The material may also be flame retardant or include flame retardant material therein to inhibit or reduce the possibility of fire.

According to one embodiment, the air enhancing disc 10 is comprised of paper or paperboard. The paper or paperboard may, however, be comprised of a single layer. The plurality of layers are typically tightly compressed to the adjacent layers so as to provide additional strength to the air enhancing disc. To provide additional absorbent capability of air enhancing material, additional layers may be added in forming the air enhancing disc. The paper or paperboard is typically comprised of a plurality of layers.

According to another embodiment, the air enhancing disc may be made from polymeric resins (plastic). The plastic air enhancing disc may include scented or fragrant portions located in the form of small spheres, balls, or the like. The fragranced portions may be located throughout the interior of the disc. For example, in FIG. 5, a plastic air enhancing disc 70 includes a plurality of fragranced spheres 72. The plastic air enhancing disc 70 may releasably engage an outer surface of a cold air inlet by, for example, a double-sided piece of tape (not shown) as described above.

According to another embodiment, an envelope with a plurality of holes formed therein (e.g., a mesh envelope or the like) may be used in the methods of the present invention. The envelope (not shown) may be formed by having two layers attached along their respective edges or along areas near the edges of the layers. The plurality of holes of the envelope are shaped and sized to permit air to pass through the cold air inlet. The envelope may include a plurality of scented beads, vitamins, and/or growth-enhancing beads therein. To inhibit the beads from exiting the envelope, the beads would preferably be larger than each of the plurality of holes of the envelope.

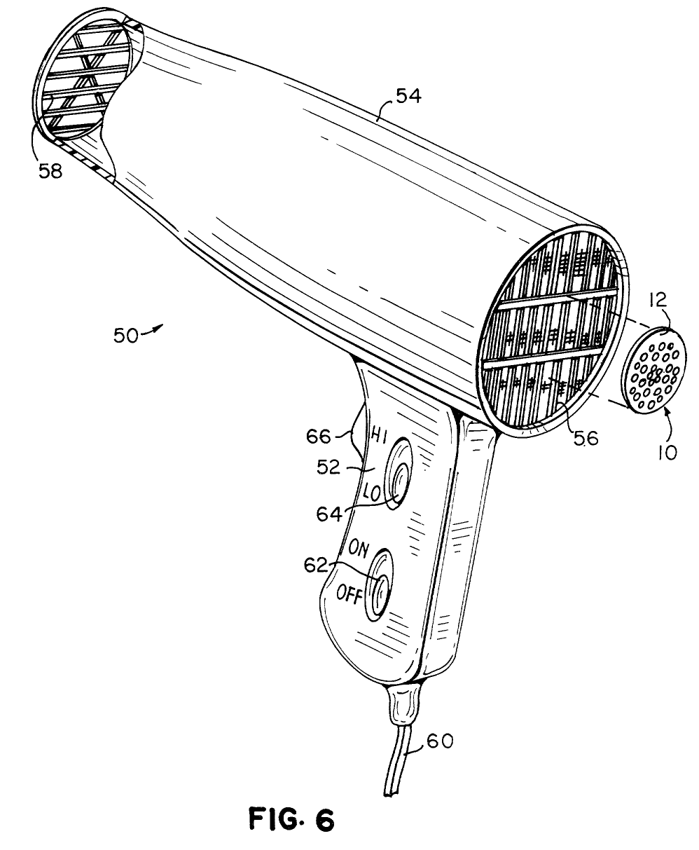

The air enhancing discs of FIGS. 1, 2 and 5 are shown as being generally circular. Other geometric shapes are contemplated, such as, but not limited to, oval, triangular, square, rectangular, hexagonal, polygonal, and the like. In addition, the air enhancing discs are not limited to geometric shapes. For example, the air enhancing discs may be shaped into non-geometric figures, such as animals, cartoon characters, and the like. The air enhancing discs may be formed in different shapes and sizes so as to approximate the shapes and sizes of different cold air inlets of different hair dryers. It is not necessary, however, to have the shape and size of the air enhancing disc exactly the same as the shape and size of the cold air inlet. For example, in FIG. 6, the air enhancing disc 10 is shaped and sized to cover a portion of the cold air inlet. If an air enhancing disc is generally circular-shaped, its diameter may be from about 16 inches, and typically is from about 1-4 inches.

The thickness of the air enhancing disc may also vary, and is generally from about 1/32-2 inches and, preferably, from about ⅛-¼ inch.

Methods of Using Air Enhancing Discs

The air enhancing discs, such as those depicted in FIGS. 1, 2 and 5, are used with hair dryers. Air enhancing discs are preferably used with a hand-held hair dryer. The hand-held hair dryer may be a conventional electric hand-held hair dryer. According to one embodiment depicted in FIG. 6, an electric hand-held hair dryer 50 includes a handle section 52 and a barrel section 54. The handle section 52 and barrel section 54 are affixed to each other. The barrel section 54 includes a generally circular-shaped cold air inlet or intake 56 and an air outlet or discharge 58. The air outlet 58 typically releases hot air, but may be selected by a user to release cold air. The hair dryer of FIG. 6 also includes an electric cord 60, an on-off switch 62, a hi-low fan speed switch 64, and a cold air trigger 66. The hair dryer includes a rotary fan (not shown) and a heating element (not shown) which respond to the selected position is of the on-off switch 62, the fan speed switch 64, and the cold air trigger 66, if applied. The details of the construction of a conventional hand-held hair dryer are well known and need not be set forth in further detail. It is contemplated that other hair dryers may be used other than the one depicted in FIG. 6.

As shown in FIG. 6, the air enhancing disc 10 is an exploded view in relation to the hair dryer 50. The air enhancing disc 10 is detachably engaged to an outer surface of the cold air inlet 56. It is contemplated that the cold air inlet 56 may be located in a different position than shown in FIG. 6. For example, the cold air inlet 56 may be located on a side of the barrel section 54. The cold air inlet 56 allows cool air to pass from the exterior of the hair dryer into the interior of the hair dryer. This cool air stream also passes through the air enhancing disc 10 before entering into the interior of the hair dryer. In using the hair dryer 50 of FIG. 6, the user selects the desired fan speed switch 64 and the “on” position of the on-off switch 62. The user grasps or holds the handle section 52 and presses the cold air trigger 66 (if cold air is desired) and directs the air stream onto the hair of a user.

The air enhancing disc 10 may be applied to the outer surface of the cold air inlet 56 during or before the operation of the hair dryer. If the air enhancing disc 10 releasably engages the outer surface solely by the pressure exerted on the second surface 14 of the disc, then the disc is preferably applied during or just before operation of the hair dryer. The air enhancing disc 10 may also be held by a user during operation of the hair dryer.

While the present invention has been described with reference to one or more particular embodiments, those skilled in the art will recognize that many changes may be made thereto without departing from the spirit and scope used in the present invention. Each of these embodiments and obvious variations thereof is contemplated as falling within the spirit and scope of the claimed invention, which is set forth in the following claims.

What is claimed is:

1. A method of using an air enhancing disc and a hair dryer, the hair dryer having a cold air inlet and an air outlet, comprising:
providing said air enhancing disc, said disc having means for allowing passage of air therethrough, said air enhancing disc having a first surface and a second surface;
releasably engaging said air enhancing disc to an outer surface of said cold air inlet;
allowing air through the means for allowing passage of air of said disc and through said cold air inlet; and
releasing a stream of enhanced air from said air outlet of the hair dryer.

2. The method of claim 1, wherein the means for allowing passage of air is a plurality of holes, said plurality of holes being formed in said air enhancing disc.

3. The method of claim 1, wherein the means for allowing passage of air is a permeable disc.

4. The method of claim 1, wherein said air enhancing disc has means for engaging the outer surface of the cold air inlet.

5. The method of claim 4, wherein the attaching means is an adhesive.

6. The method of claim 5, wherein said adhesive is selected from double-sided tape and single-sided tape.

7. The method of claim 1, wherein the step of releasably engaging the air enhancing disc to an outer surface includes the use of a clip-on structure.

8. The method of claim 1, further including a step of operating the hair dryer and wherein the step of releasably engaging the air enhancing disc to the outer surface of the cold air inlet occurs when pressure is exerted on a second surface of the air enhancing disc.

9. The method of claim 1, wherein said disc is made from a material selected from paper, paperboard, and polymeric resin.

10. The method of claim 9, wherein said disc is made from a polymeric material.

11. The method of claim 1, wherein said air enhancing disc includes perfume, cologne, scents, deodorant, or combinations thereof.

12. The method of claim 1, wherein said air enhancing disc includes vitamins.

13. The method of claim 1, wherein said air enhancing disc includes materials adapted to assist in stimulating growth or regeneration of hair.

14. The method of claim 1, wherein said air enhancing disc is an envelope.

15. A method of using an air enhancing disc and a hair dryer, the hair dryer having a cold air inlet and an air outlet, comprising:
providing said air enhancing disc, said disc having means for allowing passage of air therethrough, said air enhancing disc having a first surface and a second surface, and means for engaging the outer surface of the cold air inlet;
releasably engaging said air enhancing disc to an outer surface of the cold air inlet by the engaging means;
allowing air through the means for allowing passage of air of said disc and through the cold air inlet; and
releasing a stream of enhanced air from the air outlet of the hair dryer.

16. The method of claim 15, wherein the means for allowing passage of air is a plurality of holes, said plurality of holes being formed in the disc.

17. The method of claim 15, wherein the attaching means is an adhesive.

18. The method of claim 15, wherein the means for engaging the outer surface of the cold air inlet is a clip-on structure.

19. The method of claim 15, wherein said air enhancing disc includes perfume, cologne, scents, deodorant, or combinations thereof.

20. The method of claim 15, wherein said air enhancing disc includes materials adapted to assist in stimulating growth or regeneration of hair.
